(19) United States
(12) Patent Application Publication
(10) Pub. No.: US 2023/0293464 A1
(43) Pub. Date: Sep. 21, 2023

(54) TREATMENT OF PANCREATIC DUCTAL ADENOCARCINOMA WITH MIRDAMETINIB

(71) Applicant: SpringWorks Therapeutics Inc., Stamford, CT (US)

(72) Inventors: Badreddin Edris, Stamford, CT (US); Collin Todd, Stamford, CT (US); Todd Shearer, Stamford, CT (US); Nenad Sarapa, Stamford, CT (US); Mary Smith, Stamford, CT (US)

(21) Appl. No.: 18/185,225

(22) Filed: Mar. 16, 2023

Related U.S. Application Data
(60) Provisional application No. 63/321,045, filed on Mar. 17, 2022.

(51) Int. Cl. A61K 31/166; A61K 9/00; A61K 45/06; A61P 35/00

(52) U.S. Cl. CPC A61K 31/166; A61K 9/0053; A61K 45/06; A61P 35/00

(57) ABSTRACT

The present disclosure relates to methods for treating pancreatic ductal adenocarcinoma comprising administering to a patient in need thereof mirdametinib or a pharmaceutically acceptable salt thereof.

The present application claims the benefit of U.S. Provisional Application No. 63/321,045, filed Mar. 17, 2022, the entire contents of which is hereby incorporated by reference.

### FIELD OF THE INVENTION

The present disclosure relates to methods of treating a human patient who has pancreatic ductal adenocarcinoma (PDAC) by administering (e.g., orally) mirdametinib, or a pharmaceutically acceptable salt thereof, to the patient.

### BACKGROUND OF THE INVENTION

Pancreatic ductal adenocarcinoma (PDAC) is a highly aggressive lethal malignancy due to the lack of early diagnosis and limited response to current treatments. It is the most prevalent type of pancreatic neoplasm, and it is developed in the exocrine compartment and accounts for more than 90% of pancreatic cancer cases. Despite scientific progress on the elucidation of PDAC tumor biology and the development of novel therapeutic therapies, it has an average 5-year survival rate of less than 10%. Almost 60%-70% of PDAC cases arise from the head of the pancreas, and these cases are usually diagnosed earlier than tumors arising from the body and tail, as the head of the pancreas contains the common bile duct. Tumors of the body and tail are associated with a worse prognosis. Weight loss, abdominal pain, and jaundice are the most common symptoms observed in patients with PDAC, while less common symptoms include new-onset type 2 diabetes and thromboembolic disease. Unfortunately, the majority of patients have unresectable, locally advanced, or metastatic disease at the time of diagnosis. Moreover, traditional treatments such as chemotherapy, surgery, and radiation have not been shown to significantly improve survival. See, e.g., World. J. Gastrointest. Oncol., 12(2) 173-181, 2020.

Burmi et al., Cancer Biol Ther., 2019, 20(1):21-30, describe a study of a dual PI3k/mTOR inhibitor and MEK inhibitor PD325901 individually and together in PDAC cells.

Kashatus et al., Mol Cell., 2015, 57(3):537-51, describe experiments in which HEK-TtH, HeLa, and MPanc-96 cells were treated with the MEK inhibitor PD325901.

Wong et al., World J Gastroenterol., 2016, 22(31):7046-57, describes the signaling pathway involved in acinar-to-ductal-metaplasia which may lead to PDAC.

There is therefore a need for improved treatments of pancreatic ductal adenocarcinoma. Moreover, objective responses are not sufficient and disease recurrence after completion of therapy is common.

### BRIEF SUMMARY OF THE INVENTION

In one aspect, the present invention relates to a method of treating pancreatic ductal adenocarcinoma in a patient (e.g., a human patient in need thereof) comprising administering (e.g., orally) mirdametinib, or a pharmaceutically acceptable salt thereof, to the patient.

Another aspect is a method of treating pancreatic ductal adenocarcinoma in a patient (e.g., a human patient in need thereof) comprising administering to the patient an effective amount of a MEK inhibitor and a PIKfyve inhibitor. In one embodiment, the MEK inhibitor is selected from trametinib, cobimetinib, selumetinib, binemetinib, mirdametinib, and pharmaceutically acceptable salts thereof. In one embodiment, the MEK inhibitor is mirdametinib or a pharmaceutically acceptable salt thereof. In another embodiment, the MEK inhibitor is selumetinib or a pharmaceutically acceptable salt thereof. In one embodiment, the MEK inhibitor is orally administered. In one embodiment, the PIKfyve inhibitor is apilimod or a pharmaceutically acceptable salt thereof. In another embodiment, a synergistic effective amount of the MEK inhibitor and the PIKfyve inhibitor are administered to the patient. The MEK inhibitor and PIKfyve inhibitor may be administered by the same or different routes of administration.

Yet another aspect is a pharmaceutical composition comprising a MEK inhibitor, a PIKfyve inhibitor, and optionally one or more pharmaceutically acceptable excipients. In one embodiment, the MEK inhibitor is selected from trametinib, cobimetinib, selumetinib, binemetinib, mirdametinib, and pharmaceutically acceptable salts thereof. In one embodiment, the MEK inhibitor is mirdametinib or a pharmaceutically acceptable salt thereof. In another embodiment, the MEK inhibitor is selumetinib or a pharmaceutically acceptable salt thereof. In one embodiment, the MEK inhibitor is orally administered. In one embodiment, the PIKfyve inhibitor is apilimod or a pharmaceutically acceptable salt thereof. In another embodiment, a synergistic effective amount of the MEK inhibitor and the PIKfyve inhibitor are administered to the patient. The pharmaceutical composition can be an oral dosage form such as a tablet or capsule.

In some embodiments of any of the methods described herein, a therapeutically effective amount of mirdametinib, or a pharmaceutically acceptable salt thereof, is administered. In some embodiments of any of the methods described herein, the mirdametinib, or a pharmaceutically acceptable salt thereof, is administered in an amount of about 1 mg/m² to about 10 mg/m² per day based on mirdametinib free base. In some embodiments of any of the methods described herein, the mirdametinib, or a pharmaceutically acceptable salt thereof, is administered in an amount of about 1 mg to about 20 mg per day based on mirdametinib free base.

In some embodiments of any of the methods described herein, the mirdametinib, or a pharmaceutically acceptable salt thereof, is administered in a single dosage form comprising about 0.1 mg/m² to about 10 mg/m² based on mirdametinib free base. In some embodiments of any of the methods described herein, the mirdametinib, or a pharmaceutically acceptable salt thereof, is administered in a single dosage form comprising about 0.1 mg to about 10 mg based on mirdametinib free base.

In some embodiments of any of the methods described herein, the mirdametinib, or a pharmaceutically acceptable salt thereof, is administered once daily. In some embodiments of any of the methods described herein, the mirdametinib, or a pharmaceutically acceptable salt thereof, is administered twice daily.

In one embodiment, 1 mg/m² is orally administered twice daily to the patient. In another embodiment, 2 mg/m² is orally administered twice daily to the patient. In yet another embodiment, 3 mg/m² is orally administered twice daily to the patient.

In one embodiment of any of the methods described herein,
(a) for a patient having a body surface area between 0.38 m² and 1.80 m², the patient is initially orally administered 3.0 mg/m² mirdametinib twice daily (i.e., a total of 6 mg daily),
(b) for a patient having a body surface area between 0.44 m² and 2.22 m², the patient is initially orally administered 2.0 mg/m² mirdametinib twice daily (i.e., a total of 4 mg daily), and
(c) for a patient having a body surface area between 0.44 m² and 3.0 m², the patient is initially orally administered 1.0 mg/m² mirdametinib twice daily (i.e., a total of 2 mg daily).

In one embodiment of any of the methods described herein,
(a) for a patient having a body surface area no more than 0.69 m², the patient is initially orally administered 1 mg mirdametinib twice daily (i.e., a total of 2 mg daily),
(b) for a patient having a body surface area of 0.7 to 1.04 m², the patient is initially orally administered 2 mg mirdametinib twice daily (i.e., a total of 4 mg daily),
(c) for a patient having a body surface area of 1.05 to 1.49 m², the patient is initially orally administered 3 mg mirdametinib twice daily (i.e., a total of 6 mg daily), and
(d) for a patient having a body surface area of at least 1.5 m², the patient is initially orally administered 4 mg mirdametinib twice daily (i.e., a total of 8 mg daily).

In another embodiment of any of the methods described herein, for a patient 2 to 10 years old and having a body surface area no more than 0.69 m², the patient is initially orally administered 1 mg mirdametinib twice daily (i.e., a total of 2 mg daily). In yet another embodiment, for a patient 2 to 8 years old and having a body surface area no more than 0.69 m², the patient is initially orally administered 1 mg mirdametinib twice daily (i.e., a total of 2 mg daily). In yet another embodiment, for a patient 2 to 7 years old and having a body surface area no more than 0.69 m², the patient is initially orally administered 1 mg mirdametinib twice daily (i.e., a total of 2 mg daily). In yet another embodiment, for a patient 2 to 6 years old and having a body surface area no more than 0.69 m², the patient is initially orally administered 1 mg mirdametinib twice daily (i.e., a total of 2 mg daily). In yet another embodiment, for a patient 2 to 5 years old and having a body surface area no more than 0.69 m², the patient is initially orally administered 1 mg mirdametinib twice daily (i.e., a total of 2 mg daily).

In another embodiment of any of the methods described herein, the dose administered is reduced due to an adverse event, wherein the dose is reduced as follows:
(a) if the dose at the time of the event is 1 mg mirdametinib twice daily, then the reduced daily dose is 1 mg mirdametinib administered in the morning only;
(b) if the dose at the time of the event is 2 mg mirdametinib twice daily, then the reduced daily dose is 2 mg mirdametinib administered in the morning and 1 mg mirdametinib administered in the afternoon or evening;
(c) if the dose at the time of the event is 3 mg mirdametinib twice daily, then the reduced daily dose is 2 mg mirdametinib administered twice daily; and
(d) if the dose at the time of the event is 4 mg mirdametinib twice daily, then the reduced daily dose is 3 mg mirdametinib administered twice daily.

In one embodiment, the adverse event resulting in the dose reduction is acneiform.

In another embodiment of any of the methods described herein, the method comprises orally administering 1 mg mirdametinib twice daily (i.e., a total of 2 mg daily).

In another embodiment of any of the methods described herein, the maximum oral daily dose administered to the patient is 4 mg mirdametinib twice daily (i.e., a total of 8 mg daily).

In one embodiment of any of the methods described herein, about 2 mg/m² mirdametinib is orally administered to the patient twice daily.

In one embodiment of any of the methods described herein, over each four week period, the mirdametinib is administered for the first three weeks and discontinued for the last one week.

In some embodiments of any of the methods described herein, the mirdametinib, or a pharmaceutically acceptable salt thereof, exhibits high blood-brain-barrier penetration.

In some embodiments of any of the methods described herein, the mirdametinib, or a pharmaceutically acceptable salt thereof, is administered orally. In some embodiments of any of the methods described herein, the mirdametinib, or a pharmaceutically acceptable salt thereof, is dispersible in a potable liquid or orodispersible in a patient's saliva. In some embodiments of any of the methods described herein, the mirdametinib, or a pharmaceutically acceptable salt thereof, is administered orally as a solid dosage form. In some embodiments of any of the methods described herein, the solid dosage form is a tablet or capsule. In some embodiments of any of the methods described herein, the solid dosage form is a capsule.

In some embodiments of any of the methods described herein, the patient is a human. In some embodiments of any of the methods described herein, the human has an age of ≥2 and <25.

In some embodiments of any of the methods described herein, the human has had no prior exposure to MEK inhibitors.

In some embodiments of any of the methods described herein, the patient has one or more KRAS mutations. In some embodiments of any of the methods described herein, the one or more KRAS mutations is selected from the group consisting of KRAS G12V, KRAS G12R, KRAS G12A, KRAS G12C, KRAS G12D, KRAS Q61H, KRAS Q61L, and KRAS G13D.

In some embodiments of any of the methods described herein, the mirdametinib, or a pharmaceutically acceptable salt thereof, is administered as a monotherapy to treat pancreatic ductal adenocarcinoma. In some embodiments of any of the methods described herein, the mirdametinib, or a pharmaceutically acceptable salt thereof, is administered in combination with another active ingredient and/or surgery to treat pancreatic ductal adenocarcinoma.

In some embodiments of any of the methods described herein, the mirdametinib, or a pharmaceutically acceptable salt thereof, is administered in combination with one or more autophagy inhibitors. In some embodiments of any of the methods described herein, the one or more autophagy inhibitors is one or more selected from the group consisting of a PIKfyve inhibitor, a ULK inhibitor, chloroquine, and hydroxychloroquine. In some embodiments of any of the methods described herein, the autophagy inhibitor is a PIKfyve inhibitor. In some embodiments of any of the methods described herein, the PIKfyve inhibitor is apilimod, vacuolin-1, APY0201 ((E)-4-(5-(2-(3-methylbenzylidine)hydrazinlyl)-2-(pyridine-4-yl)pyrazolol[1,5-a]pyrimidin-7-yl)morpholine), YM201636 (6-amino-N-(3-(4-morpholinopyrido[3′,2′:4,5]furo[3,2-d]pyrimidin-2-yl)phenyl)nicotinamide), and pharmaceutically acceptable salts thereof. In some embodiments of any of the methods described herein, the PIKfyve inhibitor (“PIKfyvei”) is apilimod. In some embodiments of any of the methods described herein, the autophagy inhibitor is a ULK inhibitor. In some embodiments of any of the methods described herein, the ULK inhibitor is one or more selected from the group consisting of DCC-3116, ULK-100 ((S)—N-(3-ethyl-1,1,1-trifluoropentan-2-yl)-4-(6-(4-(2-(piperidin-1-yl)ethoxy)phenyl)pyrazolo[1,5-a]pyrimidin-3-yl)thiophene-2-carboxamide), ULK-101 ((S)—N-(1-cyclopropyl-2,2,2-trifluoroethyl)-4-(6-(4-fluorophenyl)pyrazolo[1,5-a]pyrimidin-3-yl)thiophene-2-carboxamide), SBI-0206965 (2-[[5-bromo-2-[(3,4,5-trimethoxyphenyl)amino]-4-pyrimidinyl]oxy]-N-methyl-benzamide), MRT68921 (N-[3-[[5-cyclopropyl-2-[(1,2,3,4-tetrahydro-2-methyl-6-isoquinolinyl)amino]-4-pyrimidinyl]amino]propyl]-cyclobutanecarboxamide), MRT67307 (N-(3-(5-cyclopropyl-2-(3-(morpholinomethyl)phenylamino)pyrimidin-4-ylamino)propyl)cyclobutanecarboxamide), and NVP-BEZ235 (2-methyl-2-{4-[3-methyl-2-oxo-8-(quinolin-3-yl)-2,3-dihydro-1H-imidazo[4,5-c]quinolin-1-yl]phenyl}propanenitrile).

In one aspect, the present invention relates to a method of treating pancreatic ductal adenocarcinoma in a patient (e.g., a patient in need thereof) comprising administering an effective amount of mirdametinib (e.g., administered orally), or a pharmaceutically acceptable salt thereof, and a PIKfyve inhibitor (e.g., apilimod, or a pharmaceutically acceptable salt thereof) to the patient.

### BRIEF DESCRIPTION OF THE FIGURES

FIG. 1A shows PDAC cell lines treated with the indicated concentrations of the MEK inhibitor (MEKi) mirdametinib for 24-hours. Lysates were collected and run via western blot to probe for target inhibition.

FIG. 1B shows PDAC lines stably expressing a fluorescent mCherry-EGFP-LC3B autophagy reporter construct treated with SCH772984 (an ERK inhibitor (ERKi), 1 μM) or indicated concentrations of MEKi (mirdametinib) for 24-hours. Autophagic flux was determined using flow cytometry.

FIG. 1C shows PDAC lines stably expressing a fluorescent mCherry-EGFP-LC3B autophagy reporter construct treated for 24-hours with vehicle (DMSO) or mirdametinib (MEKi, 100 nM). Live cell imaging was performed using confocal microscopy.

FIG. 1D indicates the threshold of fluorescent intensity was set to include puncta and exclude cytosolic green and background fluorescence. Green and red puncta were quantified from FIG. 1C and plotted.

FIG. 2A shows the results of PDAC cells treated with indicated concentrations of the PIKfyve inhibitor (PIKfyvei) apilimod for 24-hours. Following treatment, lysates were extracted and autophagy proteins p62 and LC3B were probed for via western blot.

FIG. 2B shows PDAC cells treated with indicated concentrations of PIKfyvei (apilimod) for 10-days, media refreshed every 3-days. Lysates were extracted and run via western blot.

FIG. 2C shows PDAC cell lines stably expressing a fluorescent mCherry-EGFP-LC3B autophagy reporter construct treated with SCH772984 (ERKi, 1 μM) alone and in combination with PIKfyvei (50 or 100 nM, apilimod) for 24-hours. Cells were collected and autophagic index was obtained via flow cytometry. Values were normalized to DMSO.

FIG. 2D shows PDAC lines stably expressing a fluorescent mCherry-EGFP-LC3B autophagy reporter construct treated with and without apilimod (PIKfyvei, 100 nM) for 24-hours.

FIG. 3A shows dose-dependent vacuole formation induced by apilimod (Pikfyvei) treatment.

FIG. 3B shows PDAC cells treated with or without apilimod (PIKfyvei, 100 nM) for 24 and 72 hours. Cells were fixed and stained with EEA1 and LAMP1 antibodies and analyzed with confocal microscopy.

FIG. 4A shows a panel of PDAC cell lines treated with increasing concentrations of mirdametinib (MEKi) for 5-days. Following treatment, cells were stained with calcein, AM and discrete object counting was performed to quantify cells and plotted as a dose-response curve.

FIG. 4B is a scheme for a panel of PDAC cell lines treated with increasing concentrations of apilimod (PIKfyvei) for 5-days. Following treatment, cells were stained with calcein AM and discrete object counting was performed to quantify cells and plotted as a dose-response curve.

FIG. 4C shows a panel of PDAC cell lines treated with mirdametinib (MEKi) or apilimod (PIKfyvei) alone and in combination for 5-days. Following treatment, cells were counted using calcein AM staining and plotted as a dose-response curve. BLISS synergy calculations were obtained based on proliferation data and represented as a heatmap below the curves (Red=synergy, white=additivity, blue=antagonism).

FIG. 4D shows PDAC cells treated for 5-days with mirdametinib (MEKi) or apilimod (PIKfyvei) alone and in combination. Apoptosis was analyzed using fluorescence activated cell sorting of Annexin-V and propidium iodide-labeled cells.

FIG. 4E shows patient-derived PDAC organoids grown in the presence of indicated MEKi and/or PIKfyvei for 7-days. 3-D CellTiter-Glo was used to monitor viability of organoid cultures and plotted as a dose-response curve. BLISS synergy calculations were performed and plotted as a heatmap under the respective organoid growth curve.

FIG. 5 shows a panel of PDAC cell lines treated with mirdametinib (MEKi) or apilimod (PIKfyvei) alone and in combination for 5-days. Following treatment, cells were counted using calcein, AM staining and plotted as a dose-response curve. BLISS synergy calculations were obtained based on proliferation data and represented as a heatmap below the curves (Red=synergy, white=additivity, blue=antagonism).

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “mirdametinib” refers to the single enantiomer N—((R)-2,3-dihydroxypropoxy)-3,4-difluoro-2-(2-fluoro-4-iodo-phenylamino)-benzamide. Mirdametinib is an allosteric, small molecule targeting mitogen-activated protein kinase kinase (MEK).

The term “MEK inhibitor” refers to a mitogen-activated protein kinase (MAPK) extracellular signal-regulated kinase (ERK) kinase (MEK) inhibitor.

The term “mg/m²” refers to the dose in milligrams per m² body surface area of the patient.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject. In one embodiment, the patient is a human patient, such as a pediatric patient.

The term “pediatric” refers to a human subject under the age of 21 years at the time of treatment. The term “pediatric” can be further divided into various subpopulations including: neonates (from birth through the first 28 days of life); infants (29 days of age to less than two years of age); young children (two years of age to less than 12 years of age); and adolescents (12 years of age through 21 years of age (up to, but not including, the twenty-second birthday)). See, e.g., Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some additional embodiments, the pediatric patient is 2 to 8 years of age, 2 to 7 years of age, 2 to 6 years of age or 2 to 5 years of age. Younger pediatric patients in particular, such as neonates, infants and young children, can have difficulty swallowing whole capsules or tablets.

The term “dispersible” as used herein refers to a composition (e.g., a tablet, powder, granules, minitablets, or pellets) which disintegrates and/or dissolves when combined with water or another potable liquid (e.g., a non-water beverage), or a subject's own saliva when placed in the subject's mouth, with or without the addition of agitation or temperature modification. In some embodiments, the dispersible composition disintegrates or dissolves within 10 minutes, 9 minutes, 8 minutes, 7 minutes, 6 minutes, 5 minutes, 4 minutes, 3 minutes, 2 minutes, or 1 minute after being combined with water or another potable liquid. Such disintegration or dissolution need not be complete. For example, a dispersible tablet may dissolve almost entirely, but some undissolved particulate matter may remain.

The term “orodispersible” refers to a composition which is capable of dissolving or disintegrating in a subject's mouth (i.e., dissolving or disintegrating in a subject's saliva) if administered orally, without a requirement of first dissolving or disintegrating in a separate container.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

In certain aspects, a subject is successfully “treated” for a tumor, according to the methods described herein if the patient shows one or more of the following: a reduction in the size of the tumor; relief of one or more symptoms associated with the specific tumor; a reduction in the volume of the tumor; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some aspects, nationally or internationally accepted standards of treatment outcomes in a given tumor can be used to determine whether an effective amount of mirdametinib meets any of these particular endpoints (e.g., CR, PFS, PR).

In certain aspects, a subject is successfully “treated” for cancer, e.g., PDAC, according to the methods described herein if the patient shows one or more of the following: a reduction in the number of or complete absence of cancer cells; relief of one or more symptoms associated with the specific cancer; reduced morbidity and mortality; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some embodiments, nationally or internationally accepted standards of treatment outcomes in a given cancer can be used to determine whether an effective amount of mirdametinib meets any of these particular endpoints (e.g., CR, PFS, PR).

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, P A, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, F L, 2004 (incorporated herein by reference).

The term “pharmaceutically-acceptable salts” refers to the relatively non-toxic, inorganic and organic acid addition salts of mirdametinib. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include, but are not limited to, the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts. See, e.g., Berge et al., “Pharmaceutical Salts”, J. Pharm. Sci., 66, 1-19, 1977.

The pharmaceutically acceptable salts of mirdametinib also include the conventional nontoxic salts or quaternary ammonium salts of the compounds, e.g., from non-toxic organic or inorganic acids. For example, such conventional nontoxic salts include those derived from inorganic acids such as, e.g., hydrochloride, hydrobromic, sulfuric, sulfamic, phosphoric, and nitric; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, and isothionic.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

### II. Methods of Treatment

Methods for treating pancreatic ductal adenocarcinoma (PDAC) comprising administering to a patient (e.g., a patient in need thereof) mirdametinib or a pharmaceutically acceptable salt thereof are provided herein.

In some embodiments of any of the methods described herein, a therapeutically effective amount of mirdametinib, or a pharmaceutically acceptable salt thereof, is administered.

In some embodiments of any of the methods described herein, the mirdametinib, or a pharmaceutically acceptable salt thereof, is administered in an amount of about 1 mg/m² to about 10 mg/m² per day based on mirdametinib free base, about 1.5 mg/m² to about 9.5 mg/m² per day based on mirdametinib free base, about 2 mg/m² to about 9 mg/m² per day based on mirdametinib free base, about 2.5 mg/m² to about 8.5 mg/m² per day based on mirdametinib free base, about 3 mg/m² to about 8 mg/m² per day based on mirdametinib free base, about 3.5 mg/m² to about 7.5 mg/m² per day based on mirdametinib free base, about 4 mg/m² to about 7 mg/m² per day based on mirdametinib free base, about 4.5 mg/m² to about 6.5 mg/m² per day based on mirdametinib free base, or about 5 mg/m² to about 6 mg/m² per day based on mirdametinib free base. In some embodiments of any of the methods described herein, the mirdametinib, or a pharmaceutically acceptable salt thereof, is administered in an amount of about 1 mg/m² per day based on mirdametinib free base, about 1.5 mg/m² per day based on mirdametinib free base, about 2 mg/m² per day based on mirdametinib free base, about 2.5 mg/m² per day based on mirdametinib free base, about 3 mg/m² per day based on mirdametinib free base, about 3.5 mg/m² per day based on mirdametinib free base, about 4 mg/m² per day based on mirdametinib free base, about 4.5 mg/m² per day based on mirdametinib free base, about 5 mg/m² per day based on mirdametinib free base, about 5.5 mg/m² per day based on mirdametinib free base, about 6 mg/m² per day based on mirdametinib free base, about 6.5 mg/m² per day based on mirdametinib free base, about 7 mg/m² per day based on mirdametinib free base, about 7.5 mg/m² per day based on mirdametinib free base, about 8 mg/m² per day based on mirdametinib free base, about 8.5 mg/m² per day based on mirdametinib free base, about 9 mg/m² per day based on mirdametinib free base, about 9.5 mg/m² per day based on mirdametinib free base, or about 10 mg/m² per day based on mirdametinib free base.

In some embodiments of any of the methods described herein, the mirdametinib, or a pharmaceutically acceptable salt thereof, is administered in an amount of about 1 mg to about 20 mg per day based on mirdametinib free base, about 1.5 mg to about 19.5 mg per day based on mirdametinib free base, about 2 mg to about 19 mg per day based on mirdametinib free base, about 2.5 mg to about 18.5 mg per day based on mirdametinib free base, about 3 mg to about 18 mg per day based on mirdametinib free base, about 3.5 mg to about 17.5 mg per day based on mirdametinib free base, about 4 mg to about 17 mg per day based on mirdametinib free base, about 4.5 mg to about 16.5 mg per day based on mirdametinib free base, about 5 mg to about 16 mg per day based on mirdametinib free base, about 5.5 mg to about 15.5 mg per day based on mirdametinib free base, about 6 mg to about 15 mg per day based on mirdametinib free base, about 6.5 mg to about 14.5 mg per day based on mirdametinib free base, about 7 mg to about 14 mg per day based on mirdametinib free base, about 7.5 mg to about 13.5 mg per day based on mirdametinib free base, about 8 mg to about 13 mg per day based on mirdametinib free base, about 8.5 mg to about 12.5 mg per day based on mirdametinib free base, about 9 mg to about 12 mg per day based on mirdametinib free base, about 9.5 mg to about 11.5 mg per day based on mirdametinib free base, or about 10 mg to about 11 mg per day based on mirdametinib free base. In some embodiments of any of the methods described herein, the mirdametinib, or a pharmaceutically acceptable salt thereof, is administered in an amount of about 1 mg per day based on mirdametinib free base, about 1.5 mg per day based on mirdametinib free base, about 2 mg per day based on mirdametinib free base, about 2.5 mg per day based on mirdametinib free base, about 3 mg per day based on mirdametinib free base, about 3.5 mg per day based on mirdametinib free base, about 4 mg per day based on mirdametinib free base, about 4.5 mg per day based on mirdametinib free base, about 5 mg per day based on mirdametinib free base, about 5.5 mg per day based on mirdametinib free base, about 6 mg per day based on mirdametinib free base, about 6.5 mg per day based on mirdametinib free base, about 7 mg per day based on mirdametinib free base, about 7.5 mg per day based on mirdametinib free base, about 8 mg per day based on mirdametinib free base, about 8.5 mg per day based on mirdametinib free base, about 9 mg per day based on mirdametinib free base, about 9.5 mg per day based on mirdametinib free base, about 10 mg per day based on mirdametinib free base, about 10.5 mg per day based on mirdametinib free base, about 11 mg per day based on mirdametinib free base, about 11.5 mg per day based on mirdametinib free base, about 12 mg per day based on mirdametinib free base, about 12.5 mg per day based on mirdametinib free base, about 13 mg per day based on mirdametinib free base, about 13.5 mg per day based on mirdametinib free base, about 14 mg per day based on mirdametinib free base, about 14.5 mg per day based on mirdametinib free base, about 15 mg per day based on mirdametinib free base, about 15.5 mg per day based on mirdametinib free base, about 16 mg per day based on mirdametinib free base, about 16.5 mg per day based on mirdametinib free base, about 17 mg per day based on mirdametinib free base, about 17.5 mg per day based on mirdametinib free base, about 18 mg per day based on mirdametinib free base, about 18.5 mg per day based on mirdametinib free base, about 19 mg per day based on mirdametinib free base, about 19.5 mg per day based on mirdametinib free base, or about 20 mg per day based on mirdametinib free base.

In some embodiments of any of the methods described herein, the mirdametinib, or a pharmaceutically acceptable salt thereof, is administered in a single dosage form comprising about 0.1 mg/m² to about 10 mg/m² based on mirdametinib free base, about 0.5 mg/m² to about 9.5 mg/m² based on mirdametinib free base, about 1 mg/m² to about 9 mg/m² based on mirdametinib free base, about 1.5 mg/m² to about 8.5 mg/m² based on mirdametinib free base, about 2 mg/m² to about 8 mg/m² based on mirdametinib free base, about 2.5 mg/m² to about 7.5 mg/m² based on mirdametinib free base, about 3 mg/m² to about 7 mg/m² based on mirdametinib free base, about 3.5 mg/m² to about 6.5 mg/m² based on mirdametinib free base, about 4 mg/m² to about 6 mg/m² based on mirdametinib free base, or about 4.5 mg/m² to about 5.5 mg/m² based on mirdametinib free base. In some embodiments of any of the methods described herein, the mirdametinib, or a pharmaceutically acceptable salt thereof, is administered in a single dosage form comprising about 0.1 mg/m² based on mirdametinib free base, about 0.2 mg/m² based on mirdametinib free base, about 0.3 mg/m² based on mirdametinib free base, about 0.4 mg/m² based on mirdametinib free base, about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some embodiments of any of the methods described herein, the mirdametinib, or a pharmaceutically acceptable salt thereof, is administered in a single dosage form comprising about 0.1 mg to about 10 mg based on mirdametinib free base, about 0.5 mg to about 9.5 mg based on mirdametinib free base, about 1 mg to about 9 mg based on mirdametinib free base, about 1.5 mg to about 8.5 mg based on mirdametinib free base, about 2 mg to about 8 mg based on mirdametinib free base, about 2.5 mg to about 7.5 mg based on mirdametinib free base, about 3 mg to about 7 mg based on mirdametinib free base, about 3.5 mg to about 6.5 mg based on mirdametinib free base, about 4 mg to about 6 mg based on mirdametinib free base, or about 4.5 mg to about 5.5 mg based on mirdametinib free base. In some embodiments of any of the methods described herein, the mirdametinib, or a pharmaceutically acceptable salt thereof, is administered in a single dosage form comprising about 0.1 mg based on mirdametinib free base, about 0.2 mg based on mirdametinib free base, about 0.3 mg based on mirdametinib free base, about 0.4 mg based on mirdametinib free base, about 0.5 mg based on mirdametinib free base, about 1 mg based on mirdametinib free base, about 1.5 mg based on mirdametinib free base, about 2 mg based on mirdametinib free base, about 2.5 mg based on mirdametinib free base, about 3 mg based on mirdametinib free base, about 3.5 mg based on mirdametinib free base, about 4 mg based on mirdametinib free base, about 4.5 mg based on mirdametinib free base, about 5 mg based on mirdametinib free base, about 5.5 mg based on mirdametinib free base, about 6 mg based on mirdametinib free base, about 6.5 mg based on mirdametinib free base, about 7 mg based on mirdametinib free base, about 7.5 mg based on mirdametinib free base, about 8 mg based on mirdametinib free base, about 8.5 mg based on mirdametinib free base, about 9 mg based on mirdametinib free base, about 9.5 mg based on mirdametinib free base, or about 10 mg based on mirdametinib free base.

In some embodiments of any of the methods described herein, the mirdametinib, or a pharmaceutically acceptable salt thereof, is administered one, two, three, or four times per day. In some embodiments of any of the methods described herein, the mirdametinib, or a pharmaceutically acceptable salt thereof, is administered once daily. In some embodiments of any of the methods described herein, the mirdametinib, or a pharmaceutically acceptable salt thereof, is administered twice daily.

In some embodiments of any of the methods described herein, the mirdametinib, or a pharmaceutically acceptable salt thereof, is administered twice daily in an amount of about 0.5 mg/m² to about 10 mg/m² based on mirdametinib free base, about 1 mg/m² to about 9.5 mg/m² based on mirdametinib free base, about 1.5 mg/m² to about 9 mg/m² based on mirdametinib free base, about 2 mg/m² to about 8.5 mg/m² based on mirdametinib free base, about 2.5 mg/m² to about 8 mg/m² based on mirdametinib free base, about 3 mg/m² to about 7.5 mg/m² based on mirdametinib free base, about 3.5 mg/m² to about 7 mg/m² based on mirdametinib free base, about 4 mg/m² to about 6.5 mg/m² based on mirdametinib free base, about 4.5 mg/m² to about 6 mg/m² based on mirdametinib free base, or about 5 mg/m² to about 6 mg/m² based on mirdametinib free base. In some embodiments of any of the methods described herein, the mirdametinib, or a pharmaceutically acceptable salt thereof, is administered twice daily in an amount of about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some embodiments of any of the methods described herein, the mirdametinib, or a pharmaceutically acceptable salt thereof, is administered twice daily in an amount of about 0.5 mg to about 10 mg based on mirdametinib free base, about 1 mg to about 9.5 mg based on mirdametinib free base, about 1.5 mg to about 9 mg based on mirdametinib free base, about 2 mg to about 8.5 mg based on mirdametinib free base, about 2.5 mg to about 8 mg based on mirdametinib free base, about 3 mg to about 7.5 mg based on mirdametinib free base, about 3.5 mg to about 7 mg based on mirdametinib free base, about 4 mg to about 6.5 mg based on mirdametinib free base, about 4.5 mg to about 6 mg based on mirdametinib free base, or about 5 mg to about 6 mg based on mirdametinib free base. In some embodiments of any of the methods described herein, the mirdametinib, or a pharmaceutically acceptable salt thereof, is administered twice daily in an amount of about 0.5 mg based on mirdametinib free base, about 1 mg based on mirdametinib free base, about 1.5 mg based on mirdametinib free base, about 2 mg based on mirdametinib free base, about 2.5 mg based on mirdametinib free base, about 3 mg based on mirdametinib free base, about 3.5 mg based on mirdametinib free base, about 4 mg based on mirdametinib free base, about 4.5 mg based on mirdametinib free base, about 5 mg based on mirdametinib free base, about 5.5 mg based on mirdametinib free base, about 6 mg based on mirdametinib free base, about 6.5 mg based on mirdametinib free base, about 7 mg based on mirdametinib free base, about 7.5 mg based on mirdametinib free base, about 8 mg based on mirdametinib free base, about 8.5 mg based on mirdametinib free base, about 9 mg based on mirdametinib free base, about 9.5 mg based on mirdametinib free base, or about 10 mg based on mirdametinib free base.

In some embodiments of any of the methods described herein, the mirdametinib, or a pharmaceutically acceptable salt thereof, is administered in a total daily dose that does not exceed about 10 mg/m² based on mirdametinib free base, about 9.5 mg/m² based on mirdametinib free base, about 9 mg/m² based on mirdametinib free base, about 8.5 mg/m² based on mirdametinib free base, about 8 mg/m² based on mirdametinib free base, about 7.5 mg/m² based on mirdametinib free base, about 7 mg/m² based on mirdametinib free base, about 6.5 mg/m² based on mirdametinib free base, about 6 mg/m² based on mirdametinib free base, about 5.5 mg/m² based on mirdametinib free base, about 5 mg/m² based on mirdametinib free base, about 4.5 mg/m² based on mirdametinib free base, about 4 mg/m² based on mirdametinib free base, about 3.5 mg/m² based on mirdametinib free base, about 3 mg/m² based on mirdametinib free base, about 2.5 mg/m² based on mirdametinib free base, about 2 mg/m² based on mirdametinib free base, or about 1.5 mg/m² based on mirdametinib free base.

In some embodiments of any of the methods described herein, the mirdametinib, or a pharmaceutically acceptable salt thereof, is administered in a total daily dose that does not exceed about 20 mg based on mirdametinib free base, about 19.5 mg based on mirdametinib free base, about 19 mg based on mirdametinib free base, about 18.5 mg based on mirdametinib free base, about 18 mg based on mirdametinib free base, about 17.5 mg based on mirdametinib free base, about 17 mg based on mirdametinib free base, about 16.5 mg based on mirdametinib free base, about 16 mg based on mirdametinib free base, about 15.5 mg based on mirdametinib free base, about 15 mg based on mirdametinib free base, about 14.5 mg based on mirdametinib free base, about 14 mg based on mirdametinib free base, about 13.5 mg based on mirdametinib free base, about 13 mg based on mirdametinib free base, about 12.5 mg based on mirdametinib free base, about 12 mg based on mirdametinib free base, about 11.5 mg based on mirdametinib free base, about 11 mg based on mirdametinib free base, about 10.5 mg based on mirdametinib free base, about 10 mg based on mirdametinib free base, about 9.5 mg based on mirdametinib free base, about 9 mg based on mirdametinib free base, about 8.5 mg based on mirdametinib free base, about 8 mg based on mirdametinib free base, about 7.5 mg based on mirdametinib free base, about 7 mg based on mirdametinib free base, about 6.5 mg based on mirdametinib free base, about 6 mg based on mirdametinib free base, about 5.5 mg based on mirdametinib free base, about 5 mg based on mirdametinib free base, about 4.5 mg based on mirdametinib free base, about 4 mg based on mirdametinib free base, about 3.5 mg based on mirdametinib free base, about 3 mg based on mirdametinib free base, about 2.5 mg based on mirdametinib free base, about 2 mg based on mirdametinib free base, or about 1.5 mg based on mirdametinib free base.

In some embodiments of any of the methods described herein, the mirdametinib, or a pharmaceutically acceptable salt thereof, is administered as mirdametinib free base.

Methods for treating pancreatic ductal adenocarcinoma (PDAC) comprising administering to a patient (e.g., a patient in need thereof) mirdametinib free base are provided herein.

In some embodiments of any of the methods described herein, a therapeutically effective amount of mirdametinib free base is administered.

In some embodiments of any of the methods described herein, the mirdametinib free base is administered in an amount of about 1 mg/m² to about 10 mg/m² per day, about 1.5 mg/m² to about 9.5 mg/m² per day, about 2 mg/m² to about 9 mg/m² per day, about 2.5 mg/m² to about 8.5 mg/m² per day, about 3 mg/m² to about 8 mg/m² per day, about 3.5 mg/m² to about 7.5 mg/m² per day, about 4 mg/m² to about 7 mg/m² per day, about 4.5 mg/m² to about 6.5 mg/m² per day, or about 5 mg/m² to about 6 mg/m² per day. In some aspects, the mirdametinib free base is administered in an amount of about 1 mg/m² per day, about 1.5 mg/m² per day, about 2 mg/m² per day, about 2.5 mg/m² per day, about 3 mg/m² per day, about 3.5 mg/m² per day, about 4 mg/m² per day, about 4.5 mg/m² per day, about 5 mg/m² per day, about 5.5 mg/m² per day, about 6 mg/m² per day, about 6.5 mg/m² per day, about 7 mg/m² per day, about 7.5 mg/m² per day, about 8 mg/m² per day, about 8.5 mg/m² per day, about 9 mg/m² per day, about 9.5 mg/m² per day, or about 10 mg/m² per day.

In some embodiments of any of the methods described herein, the mirdametinib free base is administered in an amount of about 1 mg to about 20 mg per day, about 1.5 mg to about 19.5 mg per day, about 2 mg to about 19 mg per day, about 2.5 mg to about 18.5 mg per day, about 3 mg to about 18 mg per day, about 3.5 mg to about 17.5 mg per day, about 4 mg to about 17 mg per day, about 4.5 mg to about 16.5 mg per day, about 5 mg to about 16 mg per day, about 5.5 mg to about 15.5 mg per day, about 6 mg to about 15 mg per day, about 6.5 mg to about 14.5 mg per day, about 7 mg to about 14 mg per day, about 7.5 mg to about 13.5 mg per day, about 8 mg to about 13 mg per day, about 8.5 mg to about 12.5 mg per day, about 9 mg to about 12 mg per day, about 9.5 mg to about 11.5 mg per day, or about 10 mg to about 11 mg per day. In some embodiments of any of the methods described herein, the mirdametinib free base is administered in an amount of about 1 mg per day, about 1.5 mg per day, about 2 mg per day, about 2.5 mg per day, about 3 mg per day, about 3.5 mg per day, about 4 mg per day, about 4.5 mg per day, about 5 mg per day, about 5.5 mg per day, about 6 mg per day, about 6.5 mg per day, about 7 mg per day, about 7.5 mg per day, about 8 mg per day, about 8.5 mg per day, about 9 mg per day, about 9.5 mg per day, about 10 mg per day, about 10.5 mg per day, about 11 mg per day, about 11.5 mg per day, about 12 mg per day, about 12.5 mg per day, about 13 mg per day, about 13.5 mg per day, about 14 mg per day, about 14.5 mg per day, about 15 mg per day, about 15.5 mg per day, about 16 mg per day, about 16.5 mg per day, about 17 mg per day, about 17.5 mg per day, about 18 mg per day, about 18.5 mg per day, about 19 mg per day, about 19.5 mg per day, or about 20 mg per day.

In some embodiments of any of the methods described herein, the mirdametinib free base is administered in a single dosage form comprising about 0.1 mg/m² to about 10 mg/m², about 0.5 mg/m² to about 9.5 mg/m², about 1 mg/m² to about 9 mg/m², about 1.5 mg/m² to about 8.5 mg/m², about 2 mg/m² to about 8 mg/m², about 2.5 mg/m² to about 7.5 mg/m², about 3 mg/m² to about 7 mg/m², about 3.5 mg/m² to about 6.5 mg/m², about 4 mg/m² to about 6 mg/m², or about 4.5 mg/m² to about 5.5 mg/m². In some embodiments of any of the methods described herein, the mirdametinib free base is administered in a single dosage form comprising about 0.1 mg/m², about 0.2 mg/m², about 0.3 mg/m², about 0.4 mg/m², about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m².

In some embodiments of any of the methods described herein, the mirdametinib free base is administered in a single dosage form comprising about 0.1 mg to about 10 mg, about 0.5 mg to about 9.5 mg, about 1 mg to about 9 mg, about 1.5 mg to about 8.5 mg, about 2 mg to about 8 mg, about 2.5 mg to about 7.5 mg, about 3 mg to about 7 mg, about 3.5 mg to about 6.5 mg, about 4 mg to about 6 mg, or about 4.5 mg to about 5.5 mg. In some embodiments of any of the methods described herein, the mirdametinib free base is administered in a single dosage form comprising about 0.1 mg, about 0.2 mg, about 0.3 mg, about 0.4 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some embodiments of any of the methods described herein, the mirdametinib free base is administered one, two, three, or four times per day. In some In some embodiments of any of the methods described herein, the mirdametinib free base is administered once daily. In some embodiments of any of the methods described herein, the mirdametinib free base is administered twice daily. In some embodiments of any of the methods described herein, the mirdametinib free base is administered three times daily. In some embodiments of any of the methods described herein, the mirdametinib free base is administered four times daily.

In some embodiments of any of the methods described herein, the mirdametinib free base is administered twice daily in an amount of about 0.5 mg/m² to about 10 mg/m², about 1 mg/m² to about 9.5 mg/m², about 1.5 mg/m² to about 9 mg/m², about 2 mg/m² to about 8.5 mg/m², about 2.5 mg/m² to about 8 mg/m², about 3 mg/m² to about 7.5 mg/m², about 3.5 mg/m² to about 7 mg/m², about 4 mg/m² to about 6.5 mg/m², about 4.5 mg/m² to about 6 mg/m², or about 5 mg/m² to about 6 mg/m². In some embodiments of any of the methods described herein, the mirdametinib free base is administered twice daily in an amount of about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m².

In some embodiments of any of the methods described herein, the mirdametinib free base is administered twice daily in an amount of about 0.5 mg to about 10 mg, about 1 mg to about 9.5 mg, about 1.5 mg to about 9 mg, about 2 mg to about 8.5 mg, about 2.5 mg to about 8 mg, about 3 mg to about 7.5 mg, about 3.5 mg to about 7 mg, about 4 mg to about 6.5 mg, about 4.5 mg to about 6 mg, or about 5 mg to about 6 mg. In some embodiments of any of the methods described herein, the mirdametinib free base is administered twice daily in an amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some embodiments of any of the methods described herein, the mirdametinib free base is administered in a total daily dose that does not exceed about 10 mg/m², about 9.5 mg/m², about 9 mg/m², about 8.5 mg/m², about 8 mg/m², about 7.5 mg/m², about 7 mg/m², about 6.5 mg/m², about 6 mg/m², about 5.5 mg/m², about 5 mg/m², about 4.5 mg/m², about 4 mg/m², about 3.5 mg/m², about 3 mg/m², about 2.5 mg/m², about 2 mg/m², or about 1.5 mg/m².

In some embodiments of any of the methods described herein, the mirdametinib free base is administered in a total daily dose that does not exceed about 20 mg, about 19.5 mg, about 19 mg, about 18.5 mg, about 18 mg, about 17.5 mg, about 17 mg, about 16.5 mg, about 16 mg, about 15.5 mg, about 15 mg, about 14.5 mg, about 14 mg, about 13.5 mg, about 13 mg, about 12.5 mg, about 12 mg, about 11.5 mg, about 11 mg, about 10.5 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, or about 1.5 mg.

In some embodiments of any of the methods described herein, the mirdametinib, or a pharmaceutically acceptable salt thereof, exhibits high blood-brain-barrier penetration.

In some embodiments of any of the methods described herein, the patient has one or more KRAS mutations. In some embodiments of any of the methods described herein, the one or more KRAS mutations is selected from the group consisting of KRAS G12V, KRAS G12R, KRAS G12A, KRAS G12C, KRAS G12D, KRAS Q61H, KRAS Q61L, and KRAS G13D.

In some embodiments of any of the methods described herein, the patient is a human.

In some embodiments of any of the methods described herein, the human has had no prior exposure to MEK inhibitors. In some embodiments of any of the methods described herein, the human has not responded to prior treatment to one or more MEK inhibitors.

In some embodiments of any of the methods described herein, the mirdametinib, or a pharmaceutically acceptable salt thereof, is administered orally. In some embodiments of any of the methods described herein, the mirdametinib, or a pharmaceutically acceptable salt thereof, is administered orally as a solid dosage form. In some embodiments of any of the methods described herein, the solid dosage form is a tablet or capsule. In some embodiments of any of the methods described herein, the solid dosage form is a capsule. In some embodiments of any of the methods described herein, the mirdametinib, or a pharmaceutically acceptable salt thereof, is dispersible in a potable liquid or orodispersible in a patient's saliva.

In some embodiments of any of the methods described herein, the mirdametinib, or a pharmaceutically acceptable salt thereof, is administered as a monotherapy to treat pancreatic ductal adenocarcinoma.

In some embodiments of any of the methods described herein, the mirdametinib, or a pharmaceutically acceptable salt thereof, is administered in combination with another active ingredient and/or surgery to treat pancreatic ductal adenocarcinoma. In some embodiments of any of the methods described herein, the mirdametinib, or a pharmaceutically acceptable salt thereof, is administered in combination with one or more autophagy inhibitors. In some embodiments of any of the methods described herein, the one or more autophagy inhibitor is one or more selected from the group consisting of a PIKfyve inhibitor, a ULK inhibitor, chloroquine, and hydroxychloroquine. In some embodiments of any of the methods described herein, the autophagy inhibitor is a PIKfyve inhibitor. In some embodiments of any of the methods described herein, the PIKfyve inhibitor is apilimod, vacuolin-1, APY0201 ((E)-4-(5-(2-(3-methylbenzylidine)hydrazinlyl)-2-(pyridine-4-yl)pyrazolol[1,5-a]pyrimidin-7-yl)morpholine), YM201636 (6-amino-N-(3-(4-morpholinopyrido[3′,2′:4,5]furo[3,2-d]pyrimidin-2-yl)phenyl)nicotinamide), and pharmaceutically acceptable salts thereof. In some embodiments of any of the methods described herein, the PIKfyve inhibitor is apilimod. In some embodiments of any of the methods described herein, the autophagy inhibitor is a ULK inhibitor. In some embodiments of any of the methods described herein, the ULK inhibitor is one or more selected from the group consisting of DCC-3116, ULK-100 ((S)—N-(3-ethyl-1,1,1-trifluoropentan-2-yl)-4-(6-(4-(2-(piperidin-1-yl)ethoxy)phenyl)pyrazolo[1,5-a]pyrimidin-3-yl)thiophene-2-carboxamide), ULK-101 ((S)—N-(1-cyclopropyl-2,2,2-trifluoroethyl)-4-(6-(4-fluorophenyl)pyrazolo[1,5-a]pyrimidin-3-yl)thiophene-2-carboxamide), SBI-0206965 (2-[[5-bromo-2-[(3,4,5-trimethoxyphenyl)amino]-4-pyrimidinyl]oxy]-N-methyl-benzamide), MRT68921 (N-[3-[[5-cyclopropyl-2-[(1,2,3,4-tetrahydro-2-methyl-6-isoquinolinyl)amino]-4-pyrimidinyl]amino]propyl]-cyclobutanecarboxamide), MRT67307 (N-(3-(5-cyclopropyl-2-(3-(morpholinomethyl)phenylamino)pyrimidin-4-ylamino)propyl)cyclobutanecarboxamide), and NVP-BEZ235 (2-methyl-2-{4-[3-methyl-2-oxo-8-(quinolin-3-yl)-2,3-dihydro-1H-imidazo[4,5-c]quinolin-1-yl]phenyl}propanenitrile). In some embodiments of any of the methods described herein, the autophagy inhibitor is chloroquine. In some embodiments of any of the methods described herein, the autophagy inhibitor is hydroxychloroquine.

Another aspect is a method of treating pancreatic ductal adenocarcinoma in a patient (e.g., a human patient in need thereof) comprising administering (e.g., orally) a MEK inhibitor and a PIKfyve inhibitor. The PIKfyve inhibitor may be any described herein, such as apilimod, vacuolin-1, APY0201 ((E)-4-(5-(2-(3-methylbenzylidine)hydrazinlyl)-2-(pyridine-4-yl)pyrazolol[1,5-a]pyrimidin-7-yl)morpholine), YM4201636 (6-amino-N-(3-(4-morpholinopyrido[3′,2′:4,5]furo[3,2-d]pyrimidin-2-yl)phenyl)nicotinamide), and pharmaceutically acceptable salts thereof. The MEK inhibitor may be trametinib, cobimetinib, selumetinib, binemetinib, mirdametinib, CI-1040, PD-0184264 GSK-1120212, GDC-0973, PLX-4032, AZD8330, AS-703026, RDEA-119, RO-5126766, RO-4987655, TAK-733, AS703026, PD98059, PD184352, pharmaceutically acceptable salts thereof, and any combination of any of the foregoing. In one embodiment, the MEK inhibitor is selected from trametinib, cobimetinib, selumetinib, binemetinib, mirdametinib, and pharmaceutically acceptable salts thereof.

### EXAMPLES

### Example 1: Dual Mirdametinib and Apilimod Treatment as an Efficacious Therapeutic Strategy for PDAC

Pancreatic ductal adenocarcinoma (PDAC) is characterized by KRAS- and autophagy-dependent growth. Autophagy is a multi-step, lysosomal-mediated process whereby cells degrade and recycle macromolecules to sustain growth. Inhibition of the RAF-MEK-ERK pathway resulted in upregulated autophagic flux and that dual treatment with the nonspecific autophagy inhibitor hydroxychloroquine (HCQ) and certain ERK MAPK inhibitors synergistically blocked PDAC growth. See Bryant et al., Nat Med., 2019, 25(4):628-640.

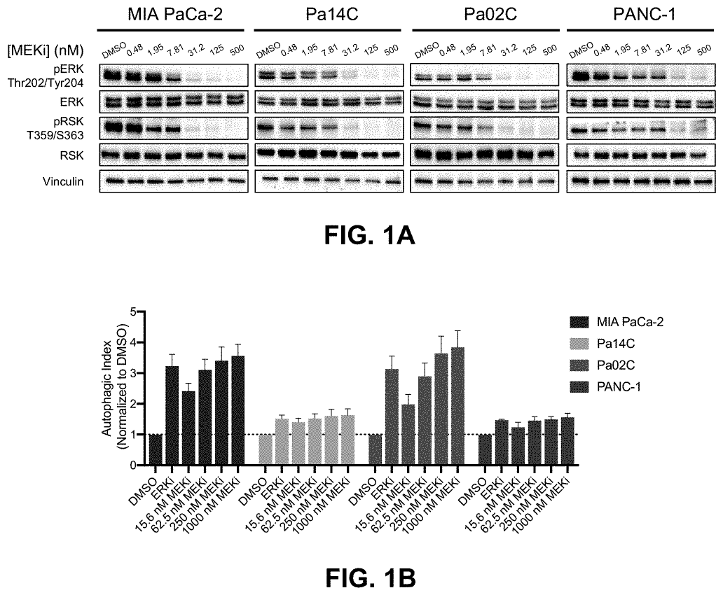

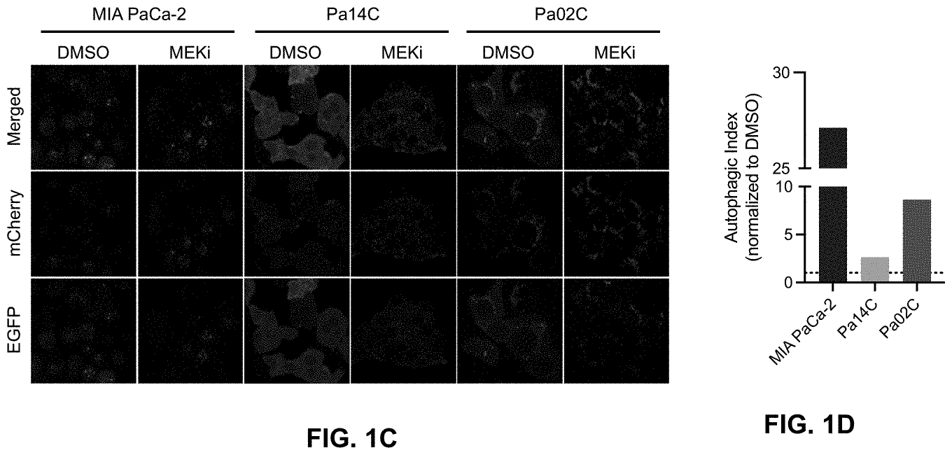

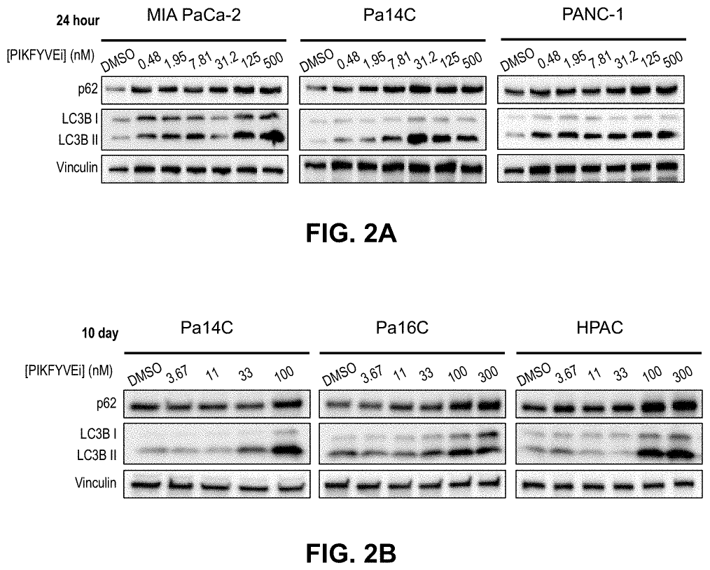

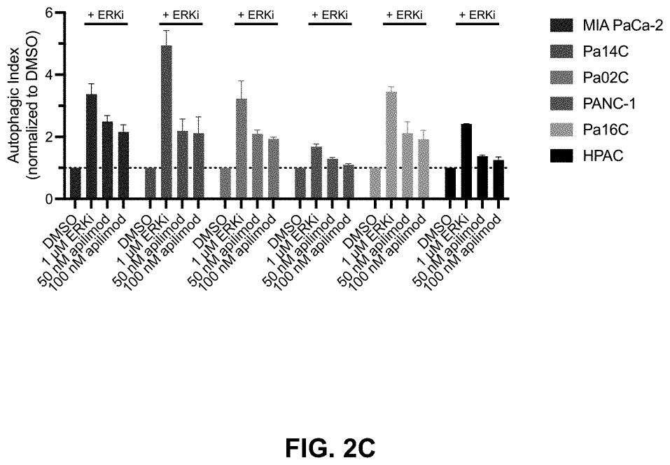

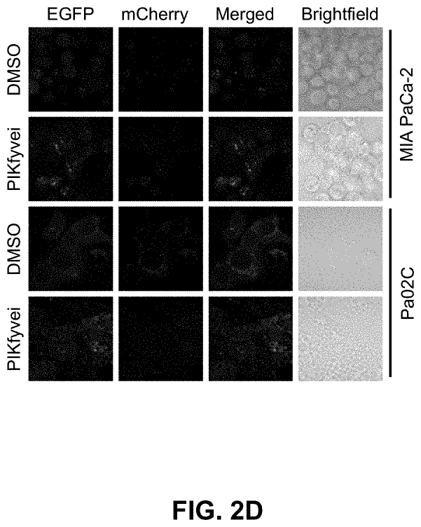

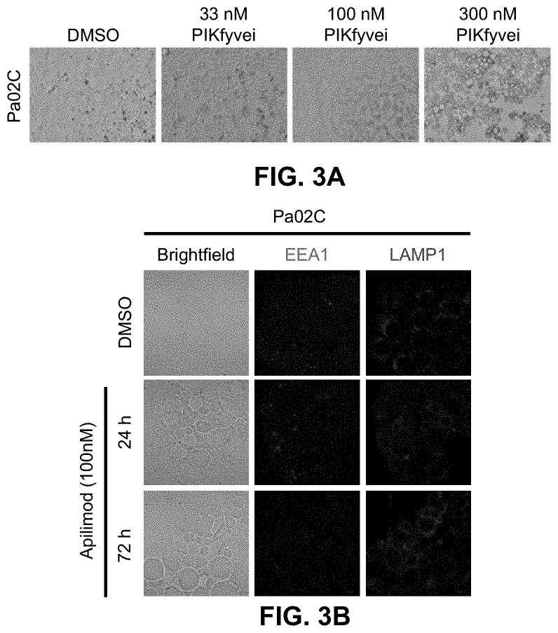

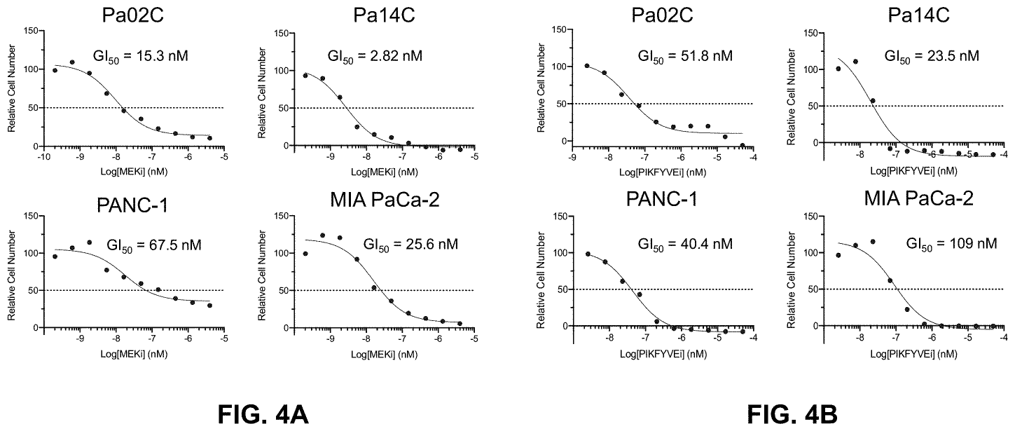

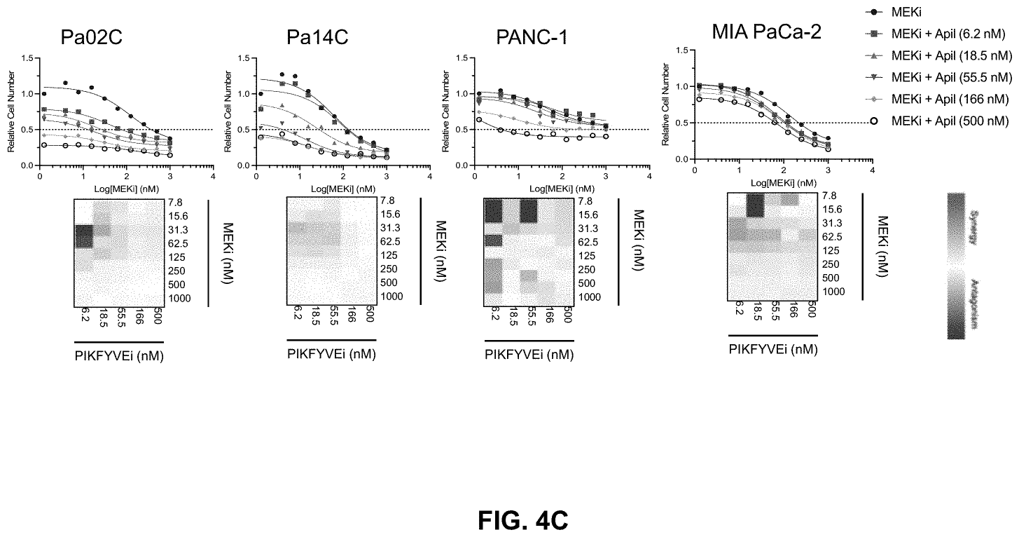

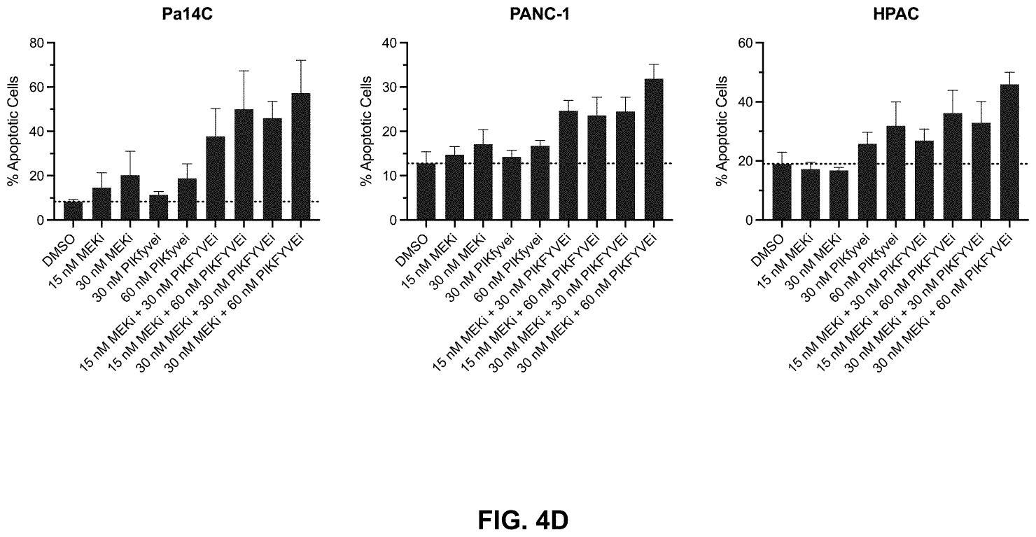

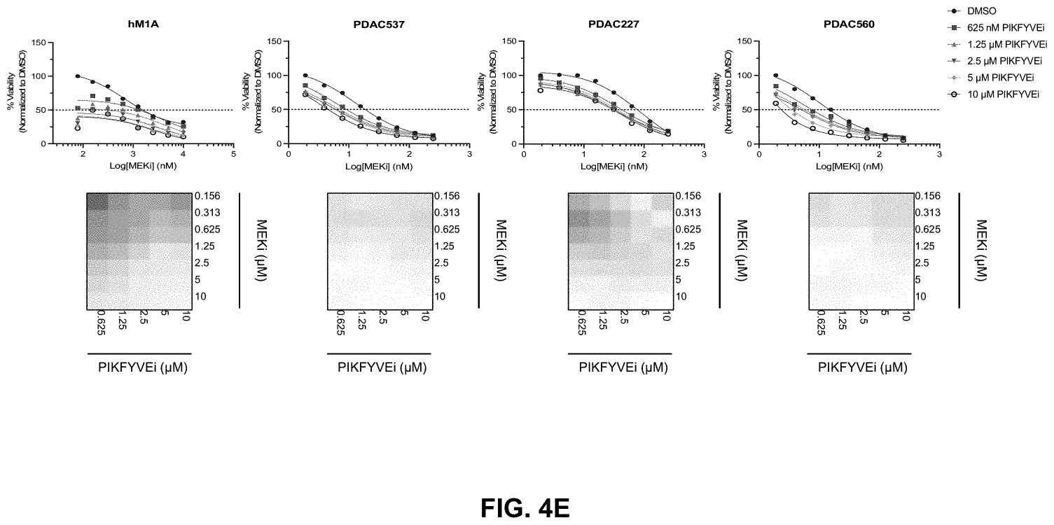

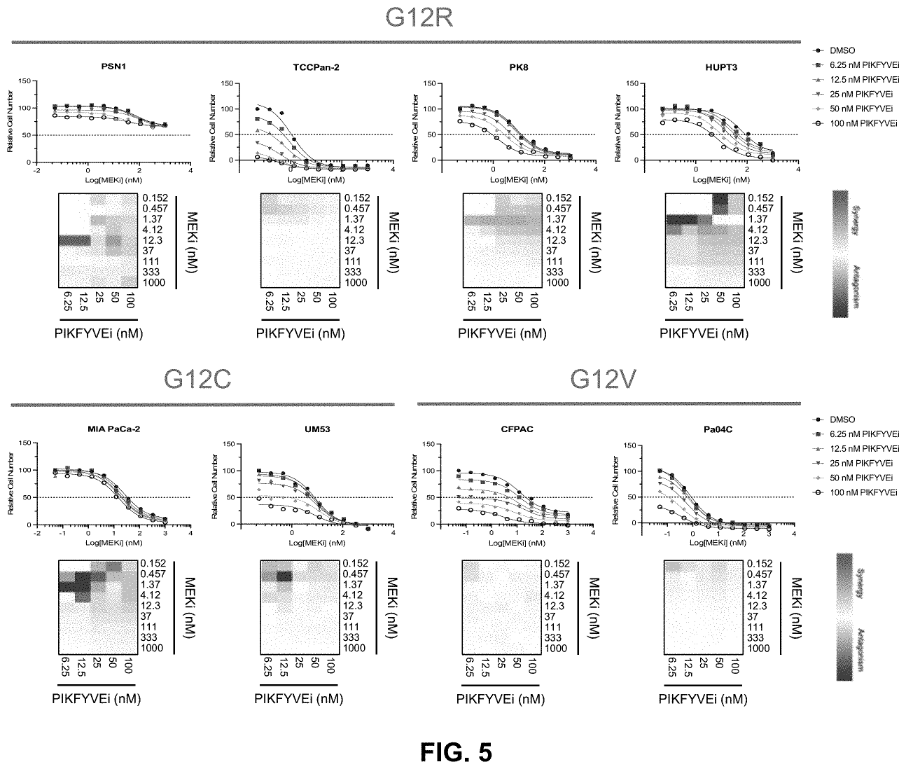

MEK inhibition with mirdametinib (PD-0325901) substantially induced autophagic flux in a panel of PDAC cell lines. See FIGS. 1A-1C. HCQ is limited in terms of specificity and potency. As exhibited in the figures, dual mirdametinib and apilimod treatment showed substantial synergy across a panel of KRAS mutant cell lines, due, in part, to a significant induction of apoptosis by the combination. See FIGS. 2A-2D, 3A-3B, 4A-4D, and 5. This synergistic relationship was maintained in patient derived PDAC organoids treated with the combination. See FIG. 4E.

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

1. A method of treating a human patient with pancreatic ductal adenocarcinoma (PDAC) comprising administering mirdametinib or a pharmaceutically acceptable salt thereof to the patient.

2. The method of claim 1, wherein a therapeutically effective amount of mirdametinib, or a pharmaceutically acceptable salt thereof, is administered.

3. The method of claim 1, wherein the mirdametinib, or a pharmaceutically acceptable salt thereof, is orally administered in an amount of about 1 mg/m² to about 10 mg/m² per day based on mirdametinib free base.

4. The method of claim 1, wherein the mirdametinib, or a pharmaceutically acceptable salt thereof, is administered in an amount of about 2 mg/m² per day based on mirdametinib free base.

5. The method of claim 1, wherein the mirdametinib, or a pharmaceutically acceptable salt thereof, is administered in an amount of about 4 mg/m² per day based on mirdametinib free base.

6. The method of claim 1, wherein the mirdametinib, or a pharmaceutically acceptable salt thereof, is administered in an amount of about 6 mg/m² per day based on mirdametinib free base.

7. The method of claim 1, wherein the mirdametinib, or a pharmaceutically acceptable salt thereof, is administered in an amount of about 1 mg to about 20 mg per day based on mirdametinib free base.

8. The method of claim 1, wherein the mirdametinib, or a pharmaceutically acceptable salt thereof, is administered once daily.

9. The method of claim 1, wherein the mirdametinib, or a pharmaceutically acceptable salt thereof, is administered twice daily.

10. The method of claim 1, wherein the method comprises orally administering to the patient 1 mg/m² mirdametinib twice daily.

11. The method of claim 1, wherein the method comprises orally administering to the patient 2 mg/m² mirdametinib twice daily.

12. The method of claim 1, wherein the method comprises orally administering to the patient 3 mg/m² mirdametinib twice daily.

13. The method of claim 1, wherein
(a) for a patient having a body surface area no more than 0.69 m², the patient is initially orally administered 1 mg mirdametinib twice daily,
(b) for a patient having a body surface area of 0.7 to 1.04 m², the patient is initially orally administered 2 mg mirdametinib twice daily,
(c) for a patient having a body surface area of 1.05 to 1.49 m², the patient is initially orally administered 3 mg mirdametinib twice daily, and
(d) for a patient having a body surface area of at least 1.5 m², the patient is initially orally administered 4 mg mirdametinib twice daily.

14. The method of claim 1, wherein
(a) for a patient having a body surface area between 0.38 m² and 1.80 m², the patient is initially orally administered 3.0 mg/m² mirdametinib twice daily,
(b) for a patient having a body surface area between 0.44 m² and 2.22 m², the patient is initially orally administered 2.0 mg/m² mirdametinib twice daily, and
(c) for a patient having a body surface area between 0.44 m² and 3.0 m², the patient is initially orally administered 1.0 mg/m² mirdametinib twice daily.

15. The method of claim 13, wherein the dose administered is reduced due to an adverse event, wherein the dose is reduced as follows:
(a) if the dose at the time of the event is 1 mg mirdametinib twice daily, then the reduced daily dose is 1 mg mirdametinib administered in the morning only;
(b) if the dose at the time of the event is 2 mg mirdametinib twice daily, then the reduced daily dose is 2 mg mirdametinib administered in the morning and 1 mg mirdametinib administered in the afternoon or evening;
(c) if the dose at the time of the event is 3 mg mirdametinib twice daily, then the reduced daily dose is 2 mg mirdametinib administered twice daily; and
(d) if the dose at the time of the event is 4 mg mirdametinib twice daily, then the reduced daily dose is 3 mg mirdametinib administered twice daily.

16. The method of claim 15, wherein the adverse event resulting in the dose reduction is acneiform.

17. The method of claim 1, wherein over each four week period, the mirdametinib is administered for the first three weeks and discontinued for the last one week.

18. The method of claim 1, wherein the patient has an age of ≥2 and <25.

19. The method of claim 1, wherein the patient has had no prior exposure to MEK inhibitors.

20. The method of claim 1, wherein the patient has one or more KRAS mutations.

21. The method of claim 20, wherein the one or more KRAS mutations is selected from the group consisting of KRAS G12V, KRAS G12R, KRAS G12A, KRAS G12C, KRAS G12D, KRAS Q61H, KRAS Q61L, and KRAS G13D.

22. The method of claim 1, wherein the mirdametinib, or a pharmaceutically acceptable salt thereof, is administered orally.

23. The method of claim 1, wherein the mirdametinib, or a pharmaceutically acceptable salt thereof, is administered as a monotherapy to treat pancreatic ductal adenocarcinoma.

24. The method of claim 1, wherein the mirdametinib, or a pharmaceutically acceptable salt thereof, is administered in combination with another active ingredient and/or surgery to treat pancreatic ductal adenocarcinoma.

25. The method of claim 1, wherein the mirdametinib, or a pharmaceutically acceptable salt thereof, is administered in combination with one or more autophagy inhibitors.

26. The method of claim 25, wherein the one or more autophagy inhibitors are selected from the group consisting of a PIKfyve inhibitor, a ULK inhibitor, chloroquine, hydroxychloroquine, and any combination of any of the foregoing.

27. The method of claim 26, wherein the autophagy inhibitor is a ULK inhibitor.

28. The method of claim 27, wherein the ULK inhibitor is selected from the group consisting of DCC-3116, ULK-100, ULK-101, SBI-0206965, MRT68921, MRT67307, and NVP-BEZ235.

29. The method of claim 1, wherein the mirdametinib, or a pharmaceutically acceptable salt thereof, is administered in combination with a PIKfyve inhibitor.

30. The method of claim 27, wherein the PIKfyve inhibitor is selected from the group consisting of apilimod, vacuolin-1, (E)-4-(5-(2-(3-methylbenzylidine)hydrazinlyl)-2-(pyridine-4-yl)pyrazolol [1,5-a]pyrimidin-7-yl)morpholine, 6-amino-N-(3-(4-morpholinopyrido[3′,2′:4,5]furo[3,2-d]pyrimidin-2-yl)phenyl)nicotinamide, and pharmaceutically acceptable salts thereof.

31-33. (canceled)
